(19) United States
(12) Patent Application Publication
(10) Pub. No.: US 2022/0306975 A1
(43) Pub. Date: Sep. 29, 2022

(54) ELECTROLYTE-CONTAINING ALCOHOLIC BEVERAGES

(71) Applicant: JHO INTELLECTUAL PROPERTY HOLDINGS, LLC, Weston, FL (US)

(72) Inventors: John H Owoc, Southwest Ranches, FL (US); Liangxi Li, Plantation, FL (US)

(21) Appl. No.: 17/707,008

(22) Filed: Mar. 29, 2022

Related U.S. Application Data
(60) Provisional application No. 63/167,359, filed on Mar. 29, 2021.

(51) Int. Cl. C12G 3/05; C12G 3/06; A61K 33/06; A61K 33/00

(52) U.S. Cl. CPC C12G 3/05; C12G 3/06; A61K 33/06; A61K 33/00

(57) ABSTRACT

A hard seltzer is made by dissolving a sodium ion source in an aqueous ethanol solution, wherein the aqueous ethanol solution comprises from 1% to 14% by volume of ethanol; dissolving a potassium ion source in the aqueous ethanol solution; dissolving a magnesium ion source in the aqueous ethanol solution; and carbonating the aqueous ethanol solution to produce the hard seltzer. The sodium ion source, the potassium ion source, and the magnesium ion source are used in amounts effective to provide a hard seltzer comprising 20 to 400 mg/serving of sodium ions, 20 to 400 mg/serving of potassium ions; and 3 to 400 mg/serving of magnesium ions.

### TECHNICAL FIELD

Various exemplary embodiments disclosed herein relate generally to alcoholic hard seltzer and other beverages for rehydration that include electrolytes.

### BACKGROUND

Alcoholic drinks have the potential to dehydrate an athlete who drinks such a beverage before or after an exercise regimen. Excessive intake of alcoholic beverages can cause headache, nausea, and vomiting. To counter these effects, alcoholic beverages with added electrolytes, e.g., sodium, counter this problem. Such electrolytes help ensure balanced muscle contraction and promotes fluid retention. By promoting fluid retention, electrolytes counter the effect of dehydration through sweating (Taylor, Bicycling, Aug. 16, 2019; URL: https://www.bicycling.com/health-nutrition/a28677144/electrolyte-beer/). Beer containing added sodium is reported to be less dehydrating than sodium-free beers when consumed during or after exercise. (Taylor, Id.; Desbrow et al., International Journal of Sport Nutrition and Exercise Metabolism, 25(3), 262-270 (2015)).

As a result, beers containing electrolyte salts and nutrients, and/or antioxidants are being marketed (Bernstein, Wine Enthusiast, Jun. 4, 2020; URL: https://www.winemag.com/2020/06/04/best-workout-beers-electrolytes/). Such beers typically contain 5% alcohol by volume or less. (Weinberg, Delish, Mar. 12, 2019; URL: https://www.delish.com/food-news/g26796640/wellness-beers-recovery-electrolytes/).

However, some athletes do not care for beer due to its “heavy” taste. Lighter-tasting alcoholic beverages for such individuals are desirable. Additionally, electrolyte-containing alcoholic beverages that have a pleasant tasting flavor such as fruit juices or other natural or artificial flavors are desirable.

Common water-based mixers or beverages include carbonated mineral water, tonic water, and club soda (Largeman-Roth, Today, Oct. 10, 2017; URL: https://www.today.com/food/-difference-between-seltzer-sparkling-water-club-soda-tonic-water-t114161). Carbonated mineral water typically contains trace amounts of natural minerals, e.g., calcium, magnesium, and sodium. A 12-ounce serving of mineral water may contain between 15 to 45 milligrams of sodium, depending on the origin and brand. Club soda is a mix of carbonated water with sodium bicarbonate, sodium chloride and potassium sulfate, and has about 95 milligrams of sodium per 12 ounce serving. Club soda does not provide appreciable amounts of alkaline earth metal salts, e.g., calcium or magnesium salts. Tonic water contains sodium salts and quinine. Quinine can cause adverse interactions with medications, so that it is undesirable in a sports drink that will be available to a wide range of consumers, some of whom might take such medications.

It is important to provide carbonated alcoholic beverages that enhance hydration when consumed before, during, or after athletic activity, which are available in a variety of flavors. These beverages exceed consumer expectations for taste, and for preventing dehydration with a beverage containing alcohol.

In view of the foregoing, it would be desirable to produce an innovative water-based alcoholic sports drink containing electrolytes. In particular, it would be desirable to produce a water-based, electrolyte rich alcoholic beverage.

### SUMMARY

In light of the present need for new and improved beverages for rehydration after athletic activity, and for general use, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a hard seltzer, including aqueous ethanol, comprising from 1% to 11% by weight of ethanol (0.8% to 14% alcohol by volume (ABV));
about 20 to about 400 mg/serving, about 30 to about 300 mg/serving, about 40 to about 250 mg/serving, about 50 to about 200 mg/serving, about 60 to about 150 mg/serving, or about 75 to about 100 mg/serving of sodium ions;
about 20 to about 400 mg/serving, about 40 to about 350 mg/serving, about 50 to about 300 mg/serving, about 75 to about 250 mg/serving, about 100 to about 200 mg/serving, or about 125 to about 150 mg/serving of potassium ions;
about 3 to about 400 mg/serving, about 3.5 to about 300 mg/serving, about 4 to about 250 mg/serving, about 5 to about 175 mg/serving, about 7 to about 80 mg/serving, about 8 to about 40 mg/serving, about 8 to about 25 mg/serving, or about 9 to about 12 mg/serving of magnesium ions;

The aqueous ethanol may be carbonated. The aqueous ethanol may contain from 3.2% to 9.5% by weight (4% to 12% ABV), from 3.55% to 7.9% by weight (4.5% to 10% ABV), from 3.95% to 7.9% by weight (5% to 10% ABV), from 1% to 7% by weight (0.8% to 8.9% ABV), from 2% to 6.5% by weight (1.6% to 8.2% ABV), from 3% to 6% by weight (2.4% to 7.6% ABV), or from 4% to 6% by weight (3.2% to 7.6% ABV) of ethanol.

The aqueous ethanol medium may contain:
1% to 7% by weight of ethanol, and 93% to 99% by weight of water;
2% to 6.5% by weight of ethanol, and 93.5% to 98% by weight of water;
3% to 6% by weight of ethanol, and 94% to 97% by weight of water; or
4% to 6% by weight of ethanol, and 94% to 96% by weight of water.

Various embodiments disclosed herein relate to a hard seltzer, comprising:
aqueous ethanol, comprising from about 0.1% to about 18% by volume of ethanol;
about 10 to about 500 mg/serving of sodium ions;
about 10 to about 750 mg/serving of potassium ions; and
about 1 to about 400 mg/serving of magnesium ions,
wherein the aqueous ethanol is carbonated or non-carbonated.

In some embodiments, the water may be replaced with a mixture of water and from 2% to 20%, from 4% to 15% from 5% to 12%, or from 5% to 10% fruit juice, based on the combined weight of the water and the juice. The sweetness and/or tartness of the resulting beverage may be adjusted with a natural or artificial sweetener, or by adjusting the volume of juice in the resulting beverage. The resulting beverage offers excellent flavor, and has a mixture of salts which are highly effective for enhancing hydration after athletic activity, limiting dehydration during athletic activity. The beverage may be drunk straight out of the bottle, or used as a highly flavorful mixer in preparation of various cocktails.

Various embodiments disclosed herein relate to an alcoholic beverage, including aqueous ethanol and sodium ions obtained from a sodium ion source. The sodium ion source may be trisodium citrate, disodium citrate, monosodium citrate, sodium chloride, sodium carbonate, sodium hydroxide, sodium bicarbonate, monosodium glutamate, hydrates thereof, or mixtures thereof.

Various embodiments disclosed herein relate to a hard seltzer, including aqueous ethanol and potassium ions obtained from a potassium ion source. The potassium ion source may be a citrate salt, potassium chloride, potassium carbonate, potassium hydroxide, potassium bicarbonate, a glutamate salt, hydrates thereof, or mixtures thereof.

Various embodiments disclosed herein relate to a hard seltzer, including aqueous ethanol and magnesium ions obtained from a magnesium ion source. The magnesium ion source may be a citrate salt, magnesium chloride, magnesium carbonate, magnesium hydroxide, magnesium bicarbonate, a glutamate salt, hydrates thereof, or mixtures thereof.

The hard seltzer may contain a sodium ion source, a potassium ion source, or a magnesium ion source. The hard seltzer may contain at least two of a sodium ion source, a potassium ion source, and a magnesium ion source. The hard seltzer may contain each of a sodium ion source, a potassium ion source, and a magnesium ion source. The hard seltzer may also contain sources of calcium ions, phosphorus, manganese ions, sulfur, chloride ions, iron ions, iodide ions, fluoride ions, zinc ions, copper ions, selenium, chromium ions, and cobalt ions, where each ion is present in an amount ranging from 1% to 100%, from 2% to 50%, from 3% to 20%, or from 4% to 10% of the recommended daily value.

The hard seltzer may further include a natural or artificial sweetener, a natural or artificial flavor, a pH adjustor, or a mixture thereof.

Various exemplary embodiments relate to a hard seltzer, including aqueous ethanol, comprising:

3.2% to 9.5% by weight (4% to 12% ABV), from 3.95% to 7.9% by weight (5% to 10% ABV), from 0.8% to 8.9% by weight (1% to 7% ABV), from 2.4% to 7.6% by weight (3% to 6% ABV), or from 3.2% to 7.6% by weight (4% to 6% ABV) of ethanol; from 1 to 10%, or from 2 to 7%, or from 3% to 5%, of the recommended daily value of sodium ions; from 0.5 to 10%, or from 1 to 5%, or from 2% to 4%, of the recommended daily value of potassium ions; and from 0.1 to 10° A, or from 0.5 to 5%, or from 1% to 3%, of the recommended daily value of magnesium ions. In various embodiments, the aqueous ethanol is carbonated. The aqueous ethanol may contain from 2% to 6.5% by weight, from 3% to 6% by weight, or from 4% to 6% by weight of ethanol.

Various embodiments relate to a method of making a hard seltzer, by dissolving a sodium ion source in an aqueous ethanol solution, wherein the aqueous ethanol solution comprises from 1% to 7% by weight of ethanol; dissolving a potassium ion source in the aqueous ethanol solution; dissolving a magnesium ion source in the aqueous ethanol solution; and carbonating the aqueous ethanol solution. The sodium ion source, the potassium ion source, and the magnesium ion source are used in amounts effective to provide a hard seltzer comprising 20 to 400 mg/serving of sodium ions; 20 to 400 mg/serving of potassium ions; and 3 to 400 mg/serving of magnesium ions.

The aqueous ethanol solution may be carbonated before addition of the sodium ion source, the potassium ion source, and the magnesium ion source to the aqueous ethanol. Alternatively, the aqueous ethanol solution may be carbonated after addition of the various ion sources to the aqueous ethanol.

Various embodiments relate to a method of making a hard seltzer, by:

producing an aqueous ethanol solution by mixing ethanol with carbonated water, wherein the aqueous ethanol solution comprises from 1% to 7% by weight of ethanol, based on the combined weight of the carbonated water and ethanol; dissolving a sodium ion source in an aqueous ethanol solution; dissolving a potassium ion source in the aqueous ethanol solution; and dissolving a magnesium ion source in the aqueous ethanol solution.

### DETAILED DESCRIPTION

As used herein, the term “about” means “±10%,” “±5%,” or “±2%.”

In the following discussion, it is understood that metal ions are added as a corresponding metal salt. The weight of metal ion added to the drink discussed herein, W_{MET}, is calculated as the weight of the salt added to the drink, W_{SALT}, multiplied by a ratio of the molar mass of the metal, MM_{MET}, to the molar mass of the salt, MM_{SALT}, as follows:

W_{MET}=W_{SALT}×(MM_{MET}/MM_{SALT})

Various embodiments disclosed herein relate to a hard seltzer, including:
carbonated or non-carbonated aqueous ethanol, comprising from about 0.1° A to about 18% by volume of ethanol;
about 10 to about 500 mg/serving of sodium ions;
about 10 to about 750 mg/serving of potassium ions; and
about 1 to about 400 mg/serving of magnesium ions.

The sodium ion source, the potassium ion source, and the magnesium ion source may be used in amounts effective to provide a hard seltzer comprising:
20 to 400 mg/serving of sodium ions; and
a combination of potassium ions and magnesium ions, where:
   a ratio of sodium ions to potassium ions is about 1:20 to about 20:1 by weight, about 1:10 to about 10:1 by weight, about 1:5 to about 5:1 by weight, about 1:3 to about 3:1 by weight, about 1:3 to about 1:1 by weight, or about 1:2 to about 2:3 by weight;
   a ratio of sodium ions to magnesium ions is about 135:1 to about 1:20 by weight, about 100:1 to about 1:15 by weight, about 75:1 to about 1:10 by weight, about 50:1 to about 1:5 by weight, about 25:1 to about 1:1 by weight, or about 10:1 to about 1:1 by weight; and/or
   a ratio of potassium ions to magnesium ions is about 135:1 to about 1:20 by weight, about 100:1 to about 1:15 by weight, about 75:1 to about 1:10 by weight, about 50:1 to about 1:5 by weight, about 25:1 to about 1:1 by weight, or about 15:1 to about 10:1 by weight.

In various embodiments, the sodium ion source, the potassium ion source, and the magnesium ion source may be used in amounts effective to provide a hard seltzer comprising:
a combination of:
   from 0.5% to 25%, from 1% to 17%, from 1 to 10%, from 2% to 12%, from 2% to 8%, or from 3% to 5% of the recommended daily value of sodium ions; and
   from 0.5% to 100%, from 1% to 50% from 1.5% to 25%, from 0.5 to 10%, or from 2% to 10% of the recommended daily value of potassium ions; and
   0.05% to 100%, from 0.1% to 50% from 0.1% to 25%, from 0.1 to 10%, or from 1% to 10% of the recommended daily value of magnesium ions.

The sodium ion source, the potassium ion source, and the magnesium ion source may be used in amounts effective to provide a hard seltzer comprising:
30 to 300 mg/serving of sodium ions; and
a combination of:
   from 0.5% to 100%, from 1% to 50% from 1.5% to 25%, or from 2% to 10% of the recommended daily value of potassium ions; and
   0.5% to 100%, from 0.6% to 50% from 0.8% to 25%, or from 1% to 10% of the recommended daily value of magnesium ions.
      In various embodiments,
a ratio of sodium ions to potassium ions is about 1:20 to about 20:1 by weight, about 1:10 to about 2010:1 by weight, about 1:5 to about 5:1 by weight, about 1:3 to about 3:1 by weight, about 1:3 to about 1:1 by weight, or about 1:2 to about 2:3 by weight; and
a ratio of sodium ions to magnesium ions is about 135:1 to about 1:20 by weight, about 100:1 to about 1:15 by weight, about 75:1 to about 1:10 by weight, about 50:1 to about 1:5 by weight, about 25:1 to about 1:1 by weight, or about 10:1 to about 1:1 by weight.

In various embodiments disclosed herein, the sodium ion source, the potassium ion source, and the magnesium ion source may be used in amounts effective to provide a hard seltzer comprising 30 to 300 mg/serving of sodium ions; 40 to 400 mg/serving of potassium ions; and 3 to 30 mg/serving of magnesium ions.

Various embodiments relate to an alcoholic beverage, including aqueous ethanol, containing from 1% to 7% by weight of ethanol; from 3% to 5% of the recommended daily value of sodium ions per serving; from 2% to 4% of the recommended daily value of potassium ions per serving; and from 1% to 3% of the recommended daily value of magnesium per serving. The aqueous ethanol may be carbonated.

The beverages disclosed herein may contain at least one natural or artificial sweetener. Suitable artificial sweeteners include Acesulfame potassium, Aspartame, Sucralose, D-Tagatose, and Saccharin. Suitable natural sweeteners include glucose, sucrose, fructose, honey, maple syrup, molasses, banana puree, date paste, date sugar, stevioside, coconut sugar, and brown rice syrup.

The beverages disclosed herein may contain at least one natural or artificial flavor. Suitable natural flavors include spices, fruit purees, fruit juices, vegetable purees, and vegetable juices.

The beverages disclosed herein may contain at least one pH adjuster. Suitable pH adjusters include acetic acid and salts thereof, adipic acid and salts thereof, sulphuric acid and salts thereof, carbonate salts, citric acid and salts thereof, hydroxide salts, phosphoric acid and salts thereof, chloride salts, lactic acid and salts thereof, calcium oxide, calcium phosphate, calcium sulphate, citric acid, potassium hydrogen tartrate, fumaric acid and salts thereof, gluconic acid and salts thereof, hydrochloric acid and salts thereof, and tartaric acid and salts thereof. In various embodiments, the pH adjuster may be a sodium, potassium, or magnesium salt of an acid, and may serve as a source of the corresponding metal ion. In various embodiments, the pH adjuster may be a pH-controlling buffer couple, including a weak acid and a sodium, potassium, or magnesium salt as a conjugate base, where the buffer performs the dual function of maintaining pH at a desired value and serving as a sodium, potassium, or magnesium ion source. Suitable buffer couples may include sodium citrate dihydrate/citric acid, potassium phosphate dibasic/potassium phosphate monobasic, or magnesium acetate/acetic acid.

### Example 1

Hard seltzer is produced using the electrolyte formulation in Table 1 below. In a 12 oz. serving of hard seltzer, the following are dissolved:
335 gm water (RO water, i.e., water purified by reverse osmosis);
22.5 gm ethanol;
375 mg trisodium citrate dihydrate, corresponding to 90 mg sodium ions;
315 mg dipotassium phosphate, corresponding to 140 mg potassium ions; and
55.5 mg trimagnesium citrate anhydrous, corresponding to 9 mg magnesium ions.
   The resulting solution of salts in aqueous ethanol is then converted into hard seltzer by dissolving carbon dioxide gas in the aqueous ethanol solution under pressure.

TABLE 1

| Electrolyte Composition | mg/12 oz | DV % |
|---|---|---|
| Sodium (from Sodium Citrate) | 90.00 | 4 |
| Potassium (from Potassium Phosphate Dibasic) | 140.00 | 3 |
| Magnesium (from Trimagnesium Citrate | 9.00 | 2 |
| Anhydrous) | | |

### Example 2

Hard seltzer is produced in a 12 oz. serving size and in a 16 oz. serving size, using the formulations presented in Table 2 below.

TABLE 2

| Ingredients | % by weight |
|---|---|
| Water | 94.40% |
| Alcohol | 5.00% |
| Sodium | 0.03% |
| Potassium | 0.04% |
| Magnesium | 0.002% |
| Sucralose | 0.04% |
| Acid | 0.42% |
| Flavor | 0.10% |

The resulting solutions of salts and sucralose in aqueous ethanol are then converted into hard seltzer by dissolving carbon dioxide gas in the aqueous ethanol solution under pressure.

Due to a high concentration of salts, the resulting beverage is highly effective for reducing the effects of dehydration due to exercise, and for promoting rehydration following exercise. Due to the reduced level of alcohol, i.e., less than about 9% by volume, the beverage has a reduced tendency toward alcohol-induced dehydration.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A hard seltzer, comprising:
aqueous ethanol, comprising from about 0.1% to about 18% by volume of ethanol;
about 10 to about 500 mg/serving of sodium ions;
about 10 to about 750 mg/serving of potassium ions; and
about 1 to about 400 mg/serving of magnesium ions.

2. The hard seltzer of claim 1, comprising:
aqueous ethanol, containing from 4% to 12% by volume of ethanol.

3. The hard seltzer of claim 1, comprising:
aqueous ethanol, containing from 2.4% to 7.6% by volume of ethanol.

4. The hard seltzer of claim 1, comprising:
aqueous ethanol, containing from 3.2% to 7.6% by volume of ethanol.

5. The hard seltzer of claim 1, wherein the sodium ions are obtained from a sodium ion source selected from the group consisting of trisodium citrate, disodium citrate, monosodium citrate, sodium chloride, sodium carbonate, sodium hydroxide, sodium bicarbonate, sodium phosphate, sodium phosphate dibasic, sodium phosphate monobasic, monosodium glutamate, hydrates thereof, and mixtures thereof.

6. The hard seltzer of claim 1, wherein the potassium ions are obtained from a potassium salt selected from the group consisting of citrate salts, phosphate salts, potassium chloride, potassium carbonate, potassium hydroxide, potassium bicarbonate, glutamate salts, hydrates thereof, and mixtures thereof.

7. The hard seltzer of claim 1, wherein the magnesium ions are obtained from a magnesium salt selected from the group consisting of citrate salts, phosphate salts, magnesium chloride, magnesium carbonate, magnesium hydroxide, magnesium bicarbonate, glutamate salts, hydrates thereof, and mixtures thereof.

8. The hard seltzer of claim 1, further comprising a natural or artificial sweetener, a natural or artificial flavor, a pH adjustor, or a mixture thereof.

9. A hard seltzer, comprising:
aqueous ethanol, comprising from 1% to 8.9% by volume of ethanol;
from 1 to 10% of the recommended daily value of sodium ions;
from 0.5 to 10% of the recommended daily value of potassium ions; and
from 0.1 to 10% of the recommended daily value of magnesium ions;
based on a single serving of the hard seltzer,
wherein the aqueous ethanol is carbonated; and
wherein the hard seltzer further comprises a pH adjuster comprising a weak acid and a conjugate base;
wherein the conjugate base is a source of the sodium, potassium, or magnesium ions.

10. The hard seltzer of claim 9, comprising:
from 3 to 5% of the recommended daily value of sodium ions;
from 2 to 4% of the recommended daily value of potassium ions; and
from 1 to 3% of the recommended daily value of magnesium.

11. The hard seltzer of claim 9, wherein the sodium ions are obtained from a sodium ion source selected from the group consisting of trisodium citrate, disodium citrate, monosodium citrate, sodium chloride, sodium carbonate, sodium hydroxide, sodium bicarbonate, and monosodium glutamate, hydrates thereof, and mixtures thereof.

12. The hard seltzer of claim 9, wherein the potassium ions are obtained from a potassium salt selected from the group consisting of citrate salts, potassium chloride, potassium carbonate, potassium hydroxide, potassium bicarbonate, glutamate salts, hydrates thereof, and mixtures thereof.

13. The hard seltzer of claim 9, wherein the magnesium ions are obtained from a magnesium salt selected from the group consisting of citrate salts, magnesium chloride, magnesium carbonate, magnesium hydroxide, magnesium bicarbonate, glutamate salts, hydrates thereof, and mixtures thereof.

14. The hard seltzer of claim 9, further comprising a natural or artificial sweetener, a natural or artificial flavor, a pH adjustor, or a mixture thereof.

15. An alcoholic beverage, comprising:
aqueous ethanol, comprising from 1% to 8.9% by volume of ethanol;
from 1 to 10% of the recommended daily value of sodium ions per serving;
from 0.5 to 10% of the recommended daily value of potassium ions per serving from 0.1 to 10% of the recommended daily value of magnesium per serving; and
an artificial sweetener selected from the group consisting of Acesulfame potassium, Aspartame, Sucralose, D-Tagatose, and Saccharin.

16. The alcoholic beverage of claim 15, wherein the artificial sweetener is Acesulfame potassium or Sucralose.

17. The alcoholic beverage of claim 15, wherein the aqueous ethanol is carbonated.

18. A method of making the hard seltzer of claim 1, comprising:
dissolving a sodium ion source in an aqueous ethanol solution, wherein the aqueous ethanol solution comprises from 1% to 8.9% by volume of ethanol;
dissolving a potassium ion source in the aqueous ethanol solution;
dissolving a magnesium ion source in the aqueous ethanol solution; and
carbonating the aqueous ethanol solution to produce the hard seltzer;
wherein the sodium ion source, the potassium ion source, and the magnesium ion source are used in amounts effective to provide a hard seltzer comprising 20 to 400 mg/serving of sodium ions, 20 to 400 mg/serving of potassium ions; and 3 to 400 mg/serving of magnesium ions.

19. A method of making the hard seltzer of claim 1, comprising:
producing an aqueous ethanol solution by mixing carbonated water with from 1% to 8.9% by volume of ethanol, based on the combined weight of the water and the ethanol;
dissolving a sodium ion source in an aqueous ethanol solution, wherein the aqueous ethanol solution comprises;
dissolving a potassium ion source in the aqueous ethanol solution; and
dissolving a magnesium ion source in the aqueous ethanol solution;
wherein the sodium ion source, the potassium ion source, and the magnesium ion source are used in amounts effective to provide a hard seltzer comprising 30 to 300 mg/serving of sodium ions, 40 to 400 mg/serving of potassium ions; and 3 to 30 mg/serving of magnesium ions.
